(19) United States
(12) Patent Application Publication
(10) Pub. No.: US 2008/0255181 A1
(43) Pub. Date: Oct. 16, 2008

(54) Methods for treating podocyte-related disorders

(75) Inventors: Jun Oh, Heidelberg (DE); Claus P. Schmitt, Heidelberg (DE)

Correspondence Address: AMGEN INC., 1120 VETERANS BOULEVARD, SOUTH SAN FRANCISCO, CA, 94080, US

(73) Assignee: University of Heidelberg, Heidelberg (DE)

(21) Appl. No.: 12/002,331

(22) Filed: Dec. 14, 2007

Related U.S. Application Data
(60) Provisional application No. 60/875,448, filed on Dec. 15, 2006.
(60) Provisional application No. 61/001,214, filed on Oct. 31, 2007.

(51) Int. Cl. A61K 31/435; A61K 31/135; A61K 31/352; A61P 13/12; A61K 31/382

(52) U.S. Cl. 514/297; 514/649; 514/454; 514/437

(57) ABSTRACT

The present invention relates to methods for treating or preventing podocyte-related diseases and disorders using compounds that modulate the calcium sensing receptor or pharmaceutical compositions comprising thereof.

### CROSS-REFERENCE TO RELATED PATENT APPLICATIONS

This application claims the benefit of priority of U.S. Provisional Patent Application No. 60/875,448, filed on Dec. 15, 2006 and of U.S. Provisional Patent Application No. 61/001,214 filed on Oct. 31, 2007.

### FIELD OF THE INVENTION

This invention relates generally to the field of medicine and, more specifically, to methods for treating or preventing disorders and diseases associated with podocyte dysfunction.

### BACKGROUND OF THE INVENTION

In a healthy kidney, the glomerular capillary wall acts as a barrier to prevent proteins from entering the urine, based on the size and electrical charge of the proteins. The filtration barrier in renal glomeruli comprises three layers: (1) a fenestrated endothelium—thin endothelial cells with 70 nm pores, filled with negatively charged glycoprotein, mostly podocalyxin; (2) a glomerular basement membrane—the specialized capillary membrane also containing negatively charged glycoproteins; (3) podocytes—the epithelial cells of Bowman's capsule, which have long projections from which foot processes arise and attach to the urinary side of the glomerular basement membrane. Foot processes from different podocytes interdigitate, leaving filtration slits of 25-65 nm between them. Across these slits, a highly organized network of several glycoproteins forms “slit pores”, through which filtration process occurs and which prevent the passage of larger molecules such as albumin. Podocytes are responsible for ˜40% of the hydraulic resistance of the filtration barrier (Drumond et al. (1994) J. Clin. Invest. 94: 1187-1195). Podocytes are the target of injury in many glomerular diseases. Their damage leads to a retraction of their foot processes and proteinuria. Laurens, W. et al. (1995) Kidney Int. 47: 1078-1086; Pavenstaadt H. et al. (1992) Br. J. Pharmacol. 107: 189-195. Further, podocyte shape changes such as retraction of foot processes and a loss of podocytes occur in minimal change and membranous nephropathy, focal segmental glomerulosclerosis (FSGS), chronic glomerulonephritis and diabetic nephropathy. Kerjaschki, D. (1997) Kidney Int. 45: 300-313; Kriz, W. et al. (1994) Kidney Int. 45: 369-376; Pagtalunan M. et al. (1997) J. Clin. Invest. 99: 342-348.

Despite many years of research, the physiological and molecular mechanisms of glomerular filtration and its disturbances are hardly understood. Thus far, the efforts of the medical community to slow down the progression of renal diseases is mainly focused on inhibitors of the renin angiotensin system the protective effect of which are limited.

### SUMMARY OF THE INVENTION

The present invention provides methods for treating or preventing diseases and disorders associated with podocyte dysfunction.

In one aspect, the invention provides methods of treating podocyte related disorders in a subject comprising administering an effective amount of a pharmaceutical composition comprising at least one calcimimetic compound together with a pharmaceutically acceptable carrier to the subject. In one aspect, the compound used to practice the methods of the invention can be a calcimimetic. In one aspect, the calcimimetic compound is a compound of the Formula I

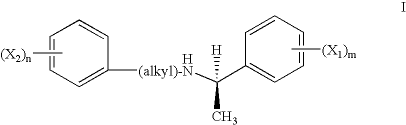

wherein X₁, X₂, n and m are as defined in Detailed Description, or a pharmaceutically acceptable salt thereof. In another aspect, the calcimimetic compound can be N-(3-[2-chlorophenyl]-propyl)-R-α-methyl-3-methoxybenzylamine or a pharmaceutically acceptable salt thereof. In a further aspect, the calcimimetic compound can be a compound of the Formula II

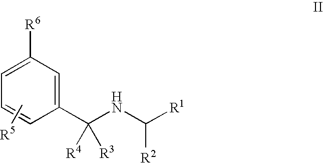

wherein R¹, R², R³, R⁴, R⁵, and R⁶ are as defined in Detailed Description, or a pharmaceutically acceptable salt thereof. In one aspect, the calcimimetic compound can be N-((6-(methyloxy)-4′-(trifluoromethyl)-1,1′-biphenyl-3-yl)methyl)-1-phenylethanamine, or a pharmaceutically acceptable salt thereof. In another aspect, the calcimimetic compound can be cinacalcet HCl.

In certain aspects of the invention the calcimimetic compound can be chosen from compounds of Formula III

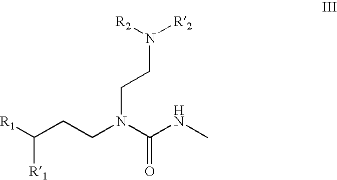

wherein R₁, R′₁, R₂, R′₂ are as detailed in the Detailed Description, or a pharmaceutically acceptable salt thereof.

The invention provides method of treating a podocyte-related disease or disorder comprising administering a therapeutically effective amount of a pharmaceutical composition comprising a calcimimetic compound together with a pharmaceutically acceptable carrier to a subject in need thereof. In one aspect, the podocyte-related disease or disorder can be podocytopenia. In another aspect, the disease or disorder can be an increase in the foot process width. In a further aspect, the podocyte-related disease or disorder can be effacement or a decrease in slit diaphragm length. In another aspect, the podocyte-related disease or disorder can be a diminution of podocyte density.

In one aspect, the podocyte-related disease or disorder can be due to a podocyte injury. The podocyte injury can be due to mechanical stress, ischemia, lack of oxygen supply, a toxic substance, an endocrinologic disorder, an infection, a contrast agent, a mechanical trauma, a cytotoxic agent, a medication, an inflammation, radiation, an infection, a dysfunction of the immune system, a genetic disorder, an organ failure, an organ transplantation, or uropathy. In one aspect, the infection can be bacterial, fungal, or viral. In one aspect, the inflammation can be due to an infection, a trauma, anoxia, obstruction, or ischemia. In one aspect, the dysfunction of the immune system can be an autoimmune disease, a systemic disease, or IgA nephropathy. In one aspect, the cytotoxic agent can be cis-platinum, adriamycin, puromycin or a calcineurin inhibitor. The medication can be an anti-bacterial, anti-viral, anti-fungal, immunosuppressive, anti-inflammatory, analgetic or anticancer agent. The ischemia is sickle-cell anemia, thrombosis, transplantation, obstruction, shock or blood loss. The genetic disorders include congenital nephritic syndrome of the Finnish type, the fetal membranous nephropathy or mutations in podocyte-specific proteins, such as α-actin-4, podocin and TRPC6. In one aspect, the podocyte-related disease or disorder can be due to an abnormal expression or function of nephrin, podocin, FAT-1, CD2AP, Neph1, integrins, integrin-linked kinase, secreted protein acid rich in cysteine, Rho GTPases, α-actinin-4, synaptopodin, cyclin-dependent kinase5, podocalyxin, hic-5, GLEPP, TRPC6, dendrin, desmin, snail, notch, synaptopodin, HSP27, lamb4, podocalyxin, NHERF2, Ezrin, α,βdystroglycans, α3 β1 integrin collagen type 4 or Wnt-4. In another aspect, the podocyte related disease or disorder can be proteinuria. Proteinuria includes microalbumiuria and macroalbumiuria. In a further aspect, the podocyte disease can be tubular atrophy.

### BRIEF DESCRIPTION OF THE DRAWINGS

FIG. 1 schematically represents calcium sensing receptor expression in proliferating and differentiated mouse podocytes and whole kidney tissue as measured by quantitative RT PCR.

FIG. 2 illustrates the expression of the CaSR in mouse podocytes as measured by Western immunoblotting.

FIG. 3 illustrates immunohistochemical staining of the calcium sensing receptor protein in differentiated mouse podocytes as seen using confocal microscopy.

FIG. 4 demonstrates that the CaSR is not found in the caveolin-1 and 2 enriched membrane fraction as indicated by Western blotting (preceded by sucrose density gradient centrifugation of mice podocyte).

FIG. 5 illustrates the time and dose dependent phosphorylation of the extra-cellular signal-regulated kinase (ERK) 1 and 2 by the calcimimetic Compound A.

FIG. 6 demonstrates that exposure of podocytes to the calcimimetic Compound A induces biphasic phosphorylation of p38 MAPK, whereas JNK is not activated in response to Compound A.

FIG. 7 illustrates induction of p90RSK and transcription factor cAMP response element-binding proteins (CREB) by the calcimimetic Compound A. C=control, R=Compound A (10 nmol/l).

FIG. 8 illustrates phosphorylation-induced inhibition of pro-apoptotic factor BAD by Compound A (at 10 nmol/l). The amount of unphosphorylated, pro-apoptotic BAD decreases with the Compound A exposure, leading to a switch to pro-survival activity of BAD. BID is not influenced by Compound A. In the graph, C=control, X-axis: time of exposure to Compound A (hours); Y-axis: ratio of phosphorylated to unphosphorylated BAD.

FIG. 9 illustrates the increased expression of Bcl-xL after exposure to Compound A. C=control, R=Compound A, numbers indicate the time of incubation in hours.

FIG. 10 illustrates phosphorylation of ERK ½ and CREB in response to Compound A in the presence of the inhibitor of MEK ½ U0126. C=Control, R=Compound A (10 nmol), I=inhibitor U0126 (10 mM), time is given in hours. U0126 strongly inhibits the calcimimetic-induced phosphorylation of ERK ½ and CREB.

FIG. 11 demonstrates that the calcimimetic Compound A prevents puromycin aminonuceloside induced apoptosis (PAN) of podocytes. Podocytes were treated with PAN (30 μg/ml), Compound A (10 nmol/l) and a combination of both for 48 h (A) and 60 h (B). The number of apoptotic cells was measured by FACS. Data from two independent experiments, each performed in duplicate, are given as percentage to medium control.

FIG. 12 demonstrates that the calcimimetic Compound A is able to prevent the development of proteinuria in rats via the binding of Compound A to the podocytes.

### DETAILED DESCRIPTION OF THE INVENTION

### I. Definitions

As used herein, the term “subject” is intended to mean a human, an aquatic mammalian or a non-aquatic animal, in need of a treatment. This subject can have, or be at risk of developing, for example, podocyte related disorders or diseases.

“Treating” or “treatment” of a disease includes: (1) preventing the disease, i.e., causing the clinical symptoms of the disease not to develop in a subject that may be or has been exposed to the disease or conditions that may cause the disease, or predisposed to the disease but does not yet experience or display symptoms of the disease, (2) inhibiting the disease, i.e., arresting or reducing the development of the disease or any of its clinical symptoms, or (3) relieving the disease, i.e., causing regression of the disease or any of its clinical symptoms.

The phrase “therapeutically effective amount” is the amount of the compound of the invention that will achieve the goal of improvement in disorder severity and the frequency of incidence. The improvement in disorder severity includes the reversal of the disease, as well as slowing down the progression of the disease.

As used herein, “calcium sensing receptor” or “CaSR” refers to the G-protein-coupled receptor responding to changes in extracellular calcium and/or magnesium levels. Activation of the CaSR produces rapid, transient increases in cytosolic calcium concentration by mobilizing calcium from thapsigargin-sensitive intracellular stores and by increasing calcium influx though voltage-insensitive calcium channels in the cell membrane (Brown et al., Nature 366: 575-580, 1993; Yamaguchi et al., Adv Pharmacol 47: 209-253, 2000).

### II. Calcimimetics Compounds and Pharmaceutical Compositions Comprising Them, Administration and Dosage

A. Calcimimetic Compounds, Definitions

As used herein, the term “calcimimetic compound” or “calcimimetic” refers to a compound that binds to calcium sensing receptors and induces a conformational change that reduces the threshold for calcium sensing receptor activation by the endogenous ligand Ca²⁺. These calcimimetic compounds can also be considered allosteric modulators of the calcium receptors.

In one aspect, a calcimimetic can have one or more of the following activities: it evokes a transient increase in internal calcium, having a duration of less that 30 seconds (for example, by mobilizing internal calcium); it evokes a rapid increase in [Ca²⁺ᵢ], occurring within thirty seconds; it evokes a sustained increase (greater than thirty seconds) in [Ca²⁺ᵢ] (for example, by causing an influx of external calcium); evokes an increase in inositol-1,4,5-triphosphate or diacylglycerol levels, usually within less than 60 seconds; and inhibits dopamine- or isoproterenol-stimulated cyclic AMP formation. In one aspect, the transient increase in [Ca²⁺ᵢ] can be abolished by pretreatment of the cell for ten minutes with 10 mM sodium fluoride or with an inhibitor of phospholipase C, or the transient increase is diminished by brief pretreatment (not more than ten minutes) of the cell with an activator of protein kinase C, for example, phorbol myristate acetate (PMA), mezerein or (−) indolactam V. In one aspect, a calcimimetic compound can be a small molecule. In another aspect, a calcimimetic can be an agonistic antibody to the CaSR.

Calcimimetic compounds useful in the present invention include those disclosed in, for example, European Patent No. 637,237, 657,029, 724,561, 787,122, 907,631, 933,354, 1,203,761, 1,235 797, 1,258,471, 1,275,635, 1,281,702, 1,284,963, 1,296,142, 1,308,436, 1,509,497, 1,509,518, 1,553,078; International Publication Nos. WO 93/04373, WO 94/18959, WO 95/11221, WO 96/12697, WO 97/41090, WO 01/34562, WO 01/90069, WO 02/14259, WO 03/099776, WO 03/099814, WO 04/017908; WO 04/094362, WO 04/106280, U.S. Pat. Nos. 5,688,938, 5,763,569, 5,962,314, 5,981,599, 6,001,884, 6,011,068, 6,031,003, 6,172,091, 6,211,244, 6,313,146, 6,342,532, 6,362,231, 6,432,656, 6,710,088, 6,750,255, 6,908,935 and U.S. Patent Application Publication No. 2002/0107406, 2003/0008876, 2003/0144526, 2003/0176485, 2003/0199497, 2004/0006130, 2004/0077619, 2005/0032796, 2005/0107448, 2005/0143426.

In certain embodiments, the calcimimetic compound is chosen from compounds of Formula I and pharmaceutically acceptable salts thereof:

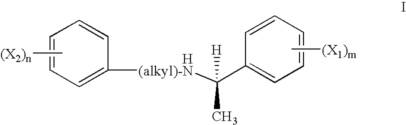

wherein:

X₁ and X₂, which may be identical or different, are each a radical chosen from CH₃, CH₃O, CH₃CH₂O, Br, Cl, F, CF₃, CHF₂, CH₂F, CF₃O, CH₃S, OH, CH₂OH, CONH₂, CN, NO₂, CH₃CH₂, propyl, isopropyl, butyl, isobutyl, t-butyl, acetoxy, and acetyl radicals, or two of X₁ may together form an entity chosen from fused cycloaliphatic rings, fused aromatic rings, and a methylene dioxy radical, or two of X₂ may together form an entity chosen from fused cycloaliphatic rings, fused aromatic rings, and a methylene dioxy radical; provided that X₂ is not a 3-t-butyl radical;

n ranges from 0 to 5;

m ranges from 1 to 5; and

the alkyl radical is chosen from C₁-C₃ alkyl radicals, which are optionally substituted with at least one group chosen from saturated and unsaturated, linear, branched, and cyclic C₁-C₉ alkyl groups, dihydroindolyl and thiodihydroindolyl groups, and 2-, 3-, and 4-piperid(in)yl groups.

The calcimimetic compound may also be chosen from compounds of Formula II:

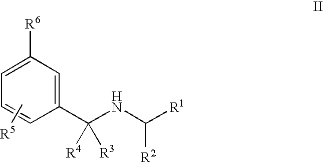

and pharmaceutically acceptable salts thereof,
wherein:

R¹ is aryl, substituted aryl, heterocyclyl, substituted heterocyclyl, cycloalkyl, or substituted cycloalkyl;

R² is alkyl or haloalkyl;

R³ is H, alkyl, or haloalkyl;

R⁴ is H, alkyl, or haloalkyl;

each R⁵ present is independently selected from the group consisting of alkyl, substituted alkyl, alkoxy, substituted alkoxy, halogen, —C(═O)OH, —CN, —NR^{d}S(═O)ₘR^{d}, —NR^{d}C(═O)NR^{d}R^{d}, —NR^{d}S(═O)ₘNR^{d}R^{d}, or —NR^{d}C(═O)R^{d};

R⁶ is aryl, substituted aryl, heterocyclyl, substituted heterocyclyl, cycloalkyl, or substituted cycloalkyl;

each R^{a} is, independently, H, alkyl or haloalkyl;

each R^{b} is, independently, aryl, aralkyl, heterocyclyl, or heterocyclylalkyl, each of which may be unsubstituted or substituted by up to 3 substituents selected from the group consisting of alkyl, halogen, haloalkyl, alkoxy, cyano, and nitro;

each R^{c} is, independently, alkyl, haloalkyl, phenyl or benzyl, each of which may be substituted or unsubstituted;

each R^{d} is, independently, H, alkyl, aryl, aralkyl, heterocyclyl, or heterocyclylalkyl wherein the alkyl, aryl, aralkyl, heterocyclyl, and heterocyclylalkyl are substituted by 0, 1, 2, 3 or 4 substituents selected from alkyl, halogen, haloalkyl, alkoxy, cyano, nitro, R^{b}, —C(═O)R^{c}, —OR^{b}, NR^{a}R^{a}, —NR^{a}R^{b}, —C(═O)OR^{c}, —C(═O)NR^{a}R^{a}, —OC(═O)R^{c}, —NR^{a}C(═O)R^{c}, —NR^{a}S(═O)ₙR^{c} and —S(═O)ₙNR^{a}R^{a};

m is 1 or 2;

n is 0, 1 or 2; and

p is 0, 1, 2, 3, or 4;

provided that if R² is methyl, p is 0, and R⁶ is unsubstituted phenyl, then R¹ is not 2,4-dihalophenyl, 2,4-dimethylphenyl, 2,4-diethylphenyl, 2,4,6-trihalophenyl, or 2,3,4-trihalophenyl. These compounds are described in detail in published US patent application number 20040082625.

In one aspect of the invention the compound of Formula II can have the formula

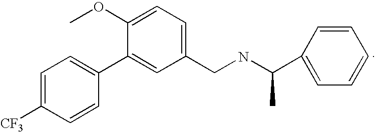

In certain aspects of the invention the calcimimetic compound can be chosen from compounds of Formula III

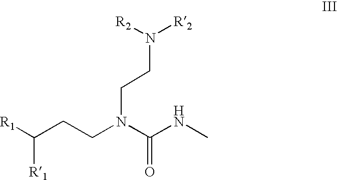

R₁ and R′₁, which may be the same or different, represent an aryl radical, a heteroaryl radical, an aryl or heteroaryl radical substituted by one or more halogen atoms, by one or more hydroxy groups, by one or more linear or branched alkyl or alkoxy radicals containing from 1 to 5 carbon atoms, by one or more trifluoromethyl, trifluoromethoxy, —CN, —NO₂, acetyl, carboxyl, carboalkoxy or thioalkyl groups and the oxidised sulfoxide or sulfone forms thereof, thiofluoroalkoxy groups,

or R₁ and R′₁ form, with the carbon atom to which they are linked, a cycle of formula:

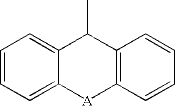

in which A represents a single bond, a —CH₂— group, an oxygen, nitrogen or sulfur atom,

R₂ and R′₂ form, with the nitrogen atom to which they are linked, a saturated heterocycle containing 4 or 5 carbon atoms optionally substituted by one or more linear or branched alkyl radicals containing from 1 to 5 carbon atoms, said heterocycle optionally containing a further heteroatom, itself being optionally substituted by a radical R₅ in which R₅ represents a hydrogen atom, a linear or branched alkyl radical containing from 1 to 5 carbon atoms, optionally substituted by an alkoxy or acyloxy radical, or R₂ and R₁₂, which may be the same or different, represent a hydrogen atom, a linear or branched alkyl radical containing from 1 to 5 carbon atoms optionally substituted by a hydroxy or alkoxy radical containing from 1 to 5 carbon atoms,

R₃ represents a thiazolyl, oxazolyl, benzothiazolyl or benzoxazolyl group of formula:

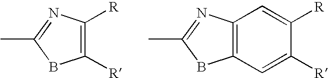

in which B represents an oxygen atom or a sulfur atom, in which R and R′, which may be the same or different, represent a hydrogen atom, a halogen atom, a hydroxy radical, a trifluoromethyl radical, a trifluoromethoxy radical, alkyl, alkoxy, alkoxycarbonyl or alkylthio radicals and the oxidised sulfoxide and sulfone form thereof linear or branched containing from 1 to 5 carbon atoms, an aryl or heteroaryl radical, an aryl or heteroaryl radical substituted by one or more groups selected from a halogen atom, a linear or branched alkyl radical containing from 1 to 5 carbon atoms, a trifluoromethyl radical, a trifluoromethoxy radical, a —CN group, an amino, dialkylamino and —NH—CO-alkyl group, an alkylthio group and the oxidised sulfoxide and sulfone form thereof, an alkylsulfonamide —NH—SO₂-alkyl group or by a morpholino group,

or R and R′ on the thiazolyl or oxazolyl group can form a saturated or unsaturated cycle comprising or not comprising one or more optionally substituted heteroatoms,

or a pharmaceutically acceptable salt thereof.

Compounds of Formula III are described in detail in the publication WO 06/117211.

In one aspect, a calcimimetic compound is N-(3-[2-chlorophenyl]-propyl)-R-▪-methyl-3-methoxybenzylamine HCl (Compound A). In another aspect, a calcimimetic compound is N-((6-(methyloxy)-4′-(trifluoromethyl)-1,1′-biphenyl-3-yl)methyl)-1-phenylethanamine (Compound B).

Calcimimetic compounds useful in the methods of the invention include the calcimimetic compounds described above, as well as their stereoisomers, enantiomers, polymorphs, hydrates, and pharmaceutically acceptable salts of any of the foregoing.

B. Methods of Assessing Calcimimetic Activity

In one aspect, compounds binding at the CaSR-activity modulating site can be identified using, for example, a labeled compound binding to the site in a competition-binding assay format.

Calcimimetic activity of a compound can be determined using techniques such as those described in International Publications WO 93/04373, WO 94/18959 and WO 95/11211.

Other methods that can be used to assess compounds' calcimimetic activity are described below.

HEK 293 Cell Assay

HEK 293 cells engineered to express human parathyroid CaSR (HEK 293 4.0-7) have been described in detail previously (Nemeth E F et al. (1998) Proc. Natl. Acad. Sci. USA 95:4040-4045). This clonal cell line has been used extensively to screen for agonists, allosteric modulators, and antagonists of the CaSR (Nemeth E F et al. (2001) J. Pharmacol. Exp. Ther. 299:323-331).

For measurements of cytoplasmic calcium concentration, the cells are recovered from tissue culture flasks by brief treatment with 0.02% ethylenediaminetetraacetic acid (EDTA) in phosphate-buffered saline (PBS) and then washed and resuspended in Buffer A (126 mM NaCl, 4 mM KCl, 1 mM CaCl₂, 1 mM MgSO₄, 0.7 mM K₂HPO₄/KH₂PO₄, 20 mM Na-Hepes, pH 7.4) supplemented with 0.1% bovine serum albumin (BSA) and 1 mg/mL D-glucose. The cells are loaded with fura-2 by incubation for 30 minutes at 37° C. in Buffer A and 2 μM fura-2 acetoxymethylester. The cells are washed with Buffer B (Buffer B is Buffer A lacking sulfate and phosphate and containing 5 mM KCl, 1 mM MgCl₂, 0.5 mM CaCl₂ supplemented with 0.5% BSA and 1 mg/ml D-glucose) and resuspended to a density of 4 to 5×10⁶ cells/ml at room temperature. For recording fluorescent signals, the cells are diluted five-fold into prewarmed (37° C.) Buffer B with constant stirring. Excitation and emission wavelengths are 340 and 510 nm, respectively. The fluorescent signal is recorded in real time using a strip-chart recorder.

For fluorometric imaging plate reader (FLIPR) analysis, HEK 293 cells are maintained in Dulbecco's modified Eagle's medium (DMEM) with 10% fetal bovine serum (FBS) and 200 μg/ml hygromycin. At 24 hrs prior to analysis, the cells are trypsinized and plated in the above medium at 1.2×10⁵ cells/well in black sided, clear-bottom, collagen 1-coated, 96-well plates. The plates are centrifuged at 1,000 rpm for 2 minutes and incubated under 5% CO₂ at 37° C. overnight. Cells are then loaded with 6 μM fluo-3 acetoxymethylester for 60 minutes at room temperature. All assays are performed in a buffer containing 126 mM NaCl, 5 mM KCl, 1 mM MgCl₂, 20 mM Na-Hepes, supplemented with 1.0 mg/ml D-glucose and 1.0 mg/ml BSA fraction IV (pH 7.4).

In one aspect, the EC₅₀'s for the CaSR-active compounds can be determined in the presence of 1 mM Ca²⁺. The EC₅₀ for cytoplasmic calcium concentration can be determined starting at an extracellular Ca²⁺ level of 0.5 mM. FLIPR experiments are done using a laser setting of 0.8 W and a 0.4 second CCD camera shutter speed. Cells are challenged with calcium, CaSR-active compound or vehicle (20 μl) and fluorescence monitored at 1 second intervals for 50 seconds. Then a second challenge (50 μl) of calcium, CaSR-active compound, or vehicle can be made and the fluorescent signal monitored. Fluorescent signals are measured as the peak height of the response within the sample period. Each response is then normalized to the maximum peak observed in the plate to determine a percentage maximum fluorescence.

Bovine Parathyroid Cells

The effect of calcimimetic compounds on CaSR-dependent regulation of PTH secretion can be assessed using primary cultures of dissociated bovine parathyroid cells. Dissociated cells can be obtained by collagenase digestion, pooled, then resuspended in Percoll purification buffer and purified by centrifugation at 14,500×g for 20 minutes at 4° C. The dissociated parathyroid cells are removed and washed in a 1:1 mixture of Ham's F-12 and DMEM (F-12/DMEM) supplemented with 0.5% BSA, 100 U/ml penicillin, 100 μg/ml streptomycin, and 20 μg/ml gentamicin. The cells are finally resuspended in F-12/DMEM containing 10 U/ml penicillin, 10 μg/ml streptomycin, and 4 μg/ml gentamicin, and BSA was substituted with ITS+(insulin, transferrin, selenous acid, BSA, and linoleic acid; Collaborative Research, Bedford, Mass.). Cells are incubated in T-75 flasks at 37° C. in a humidified atmosphere of 5% CO₂ in air.

Following overnight culture, the cells are removed from flasks by decanting and washed with parathyroid cell buffer (126 mM NaCl, 4 mM KCl, 1 mM MgSO₄, 0.7 mM K₂HPO₄/KH₂PO₄, 20 mM Na-Hepes, 20; pH 7.45 and variable amounts of CaCl₂ as specified) containing 0.1% BSA and 0.5 mM CaCl₂. The cells are resuspended in this same buffer and portions (0.3 ml) are added to polystyrene tubes containing appropriate controls, CaSR-active compound, and/or varying concentrations of CaCl₂. Each experimental condition is performed in triplicate. Incubations at 37° C. are for 20 minutes and can be terminated by placing the tubes on ice. Cells are pelleted by centrifugation (1500×g for 5 minutes at 4° C.) and 0.1 ml of supernatant is assayed immediately. A portion of the cells is left on ice during the incubation period and then processed in parallel with other samples. The amount of PTH in the supernatant from tubes maintained on ice is defined as “basal release” and subtracted from other samples. PTH is measured according to the vendor's instructions using rat PTH-(1-34) immunoradiometric assay kit (Immunotopics, San Clemente, Calif.).

MTC₆₋₂₃ Cell Calcitonin Release Rat MTC₆₋₂₃ cells (clone 6), purchased from ATCC (Manassas, Va.) are maintained in growth media (DMEM high glucose with calcium/15% HIHS) that is replaced every 3 to 4 days. The cultures are passaged weekly at a 1:4 split ratio. Calcium concentration in the formulated growth media is calculated to be 3.2 mM. Cells are incubated in an atmosphere of 90% O₂/10% CO₂, at 37° C. Prior to the experiment, cells from sub-confluent cultures are aspirated and rinsed once with trypsin solution. The flasks are aspirated again and incubated at room temperature with fresh trypsin solution for 5-10 minutes to detach the cells. The detached cells are suspended at a density of 3.0×10⁵ cells/mL in growth media and seeded at a density of 1.5×10⁵ cells/well (0.5 mL cell suspension) in collagen-coated 48 well plates (Becton Dickinson Labware, Bedford, Mass.). The cells are allowed to adhere for 56 hours post-seeding, after which the growth media was aspirated and replaced with 0.5 mL of assay media (DMEM high glucose without/2% FBS). The cells are then incubated for 16 hours prior to determination of calcium-stimulated calcitonin release. The actual calcium concentration in this media is calculated to be less than 0.07 mM. To measure calcitonin release, 0.35 mL of test agent in assay media is added to each well and incubated for 4 hours prior to determination of calcitonin content in the media. Calcitonin levels are quantified according to the vendor's instructions using a rat calcitonin immunoradiometric assay kit (Immutopics, San Clemente, Calif.).

Inositol phosphate Assay

The calcimimetic properties of compounds could also be evaluated in a biochemical assay performed on Chinese hamster ovarian (CHO) cells transfected with an expression vector containing cloned CaSR from rat brain [CHO(CaSR)] or not [CHO(WT)] (Ruat M., Snowman A M., J. Biol. Chem. 271, 1996, p 5972). CHO(CaSR) has been shown to stimulate tritiated inositol phosphate ([³H]IP) accumulation upon activation of the CaSR by Ca²⁺ and other divalent cations and by NPS 568 (Ruat et al., J. Biol. Chem. 271, 1996). Thus, [³H]IP accumulation produced by 10 μM of each CaSR-active compound in the presence of 2 mM extracellular calcium can be measured and compared to the effect produced by 10 mM extracellular calcium, a concentration eliciting maximal CaSR activation (Dauban P. et al., Bioorganic & Medicinal Chemistry Letters, 10, 2000, p 2001).

C. Pharmaceutical Compositions and Administration

Calcimimetic compounds useful in the present invention can be used in the form of pharmaceutically acceptable salts derived from inorganic or organic acids. The salts include, but are not limited to, the following: acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemisulfate, heptanoate, hexanoate, fumarate, hydrochloride, hydrobromide, hydroiodide, 2-hydroxy-ethanesulfonate, lactate, maleate, mandelate, methansulfonate, nicotinate, 2-naphthalenesulfonate, oxalate, palmoate, pectinate, persulfate, 2-phenylpropionate, picrate, pivalate, propionate, salicylate, succinate, sulfate, tartrate, thiocyanate, tosylate, mesylate, and undecanoate. When compounds of the invention include an acidic function such as a carboxy group, then suitable pharmaceutically acceptable salts for the carboxy group are well known to those skilled in the art and include, for example, alkaline, alkaline earth, ammonium, quaternary ammonium cations and the like. For additional examples of “pharmacologically acceptable salts,” see Berge et al. J. Pharm. Sci. 66: 1, 1977. In certain embodiments of the invention salts of hydrochloride and salts of methanesulfonic acid can be used.

In some aspects of the present invention, the calcium-receptor active compound can be chosen from cinacalcet, i.e., N-(1-(R)-(1-naphthyl)ethyl]-3-[3-(trifluoromethyl)phenyl]-1-aminopropane, cinacalcet HCl, and cinacalcet methanesulfonate. The calcimimetic compound, such as cinacalcet HCl and cinacalcet methanesulfonate, can be in various forms such as amorphous powders, crystalline powders, and mixtures thereof. The crystalline powders can be in forms including polymorphs, psuedopolymorphs, crystal habits, micromeretics, and particle morphology.

For administration, the compounds useful in this invention are ordinarily combined with one or more adjuvants appropriate for the indicated route of administration. The compounds may be admixed with lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinyl-pyrrolidine, and/or polyvinyl alcohol, and tableted or encapsulated for conventional administration. Alternatively, the compounds useful in this invention may be dissolved in saline, water, polyethylene glycol, propylene glycol, ethanol, corn oil, peanut oil, cottonseed oil, sesame oil, tragacanth gum, and/or various buffers. Other adjuvants and modes of administration are well known in the pharmaceutical art. The carrier or diluent may include time delay material, such as glyceryl monostearate or glyceryl distearate alone or with a wax, or other materials well known in the art.

The pharmaceutical compositions may be made up in a solid form (including granules, powders or suppositories) or in a liquid form (e.g., solutions, suspensions, or emulsions). The pharmaceutical compositions may be subjected to conventional pharmaceutical operations such as sterilization and/or may contain conventional adjuvants, such as preservatives, stabilizers, wetting agents, emulsifiers, buffers etc.

Solid dosage forms for oral administration may include capsules, tablets, pills, powders, and granules. In such solid dosage forms, the active compound may be admixed with at least one inert diluent such as sucrose, lactose, or starch. Such dosage forms may also comprise, as in normal practice, additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting, sweetening, flavoring, and perfuming agents.

The therapeutically effective amount of the calcium receptor-active compound in the compositions useful in the invention can range from about 0.1 mg to about 180 mg, for example from about 5 mg to about 180 mg, or from about 1 mg to about 100 mg of the calcimimetic compound per subject. In some aspects, the therapeutically effective amount of calcium receptor-active compound in the composition can be chosen from about 0.1 mg, about 1 mg, 5 mg, about 15 mg, about 20 mg, about 30 mg, about 50 mg, about 60 mg, about 75 mg, about 90 mg, about 120 mg, about 150 mg, about 180 mg.

While it may be possible to administer a calcium receptor-active compound to a subject alone, the compound administered will normally be present as an active ingredient in a pharmaceutical composition. Thus, a pharmaceutical composition of the invention may comprise a therapeutically effective amount of at least one calcimimetic compound, or an effective dosage amount of at least one calcimimetic compound.

As used herein, an “effective dosage amount” is an amount that provides a therapeutically effective amount of the calcium receptor-active compound when provided as a single dose, in multiple doses, or as a partial dose. Thus, an effective dosage amount of the calcium receptor-active compound of the invention includes an amount less than, equal to or greater than an effective amount of the compound; for example, a pharmaceutical composition in which two or more unit dosages, such as in tablets, capsules and the like, are required to administer an effective amount of the compound, or alternatively, a multidose pharmaceutical composition, such as powders, liquids and the like, in which an effective amount of the calcimimetic compound is administered by administering a portion of the composition.

Alternatively, a pharmaceutical composition in which two or more unit dosages, such as in tablets, capsules and the like, are required to administer an effective amount of the calcium receptor-active compound may be administered in less than an effective amount for one or more periods of time (e.g., a once-a-day administration, and a twice-a-day administration), for example to ascertain the effective dose for an individual subject, to desensitize an individual subject to potential side effects, to permit effective dosing readjustment or depletion of one or more other therapeutics administered to an individual subject, and/or the like.

The effective dosage amount of the pharmaceutical composition useful in the invention can range from about 1 mg to about 360 mg from a unit dosage form, for example about 5 mg, about 15 mg, about 30 mg, about 50 mg, about 60 mg, about 75 mg, about 90 mg, about 120 mg, about 150 mg, about 180 mg, about 210 mg, about 240 mg, about 300 mg, or about 360 mg from a unit dosage form.

In some aspects of the present invention, the compositions disclosed herein comprise a therapeutically effective amount of a calcium receptor-active compound for the treatment or prevention of diarrhea. For example, in certain embodiments, the calcimimetic compound such as cinacalcet HCl can be present in an amount ranging from about 1% to about 70%, such as from about 5% to about 40%, from about 10% to about 30%, or from about 15% to about 20%, by weight relative to the total weight of the composition.

The compositions useful in the invention may contain one or more active ingredients in addition to the calcium sensing receptor-active compound. The additional active ingredient may be another calcimimetic compound, or it may be an active ingredient having a different therapeutic activity. Examples of such additional active ingredients include renin blocker, angiotensin-converting-enzyme-inhibitor, angiotensin receptor blocker, lipid lowering agents, steroids, immunosuppressive agents, or antibiotics. When administered as a combination, the therapeutic agents can be formulated as separate compositions that are given at the same time or different times, or the therapeutic agents can be given as a single composition.

### III. Methods of Treatment

In one aspect, the invention provides methods for treatment of podocyte-related disorders or diseases. For the purposes of this invention, the terms “podocyte disease(s)” and “podocyte disorder(s)” are interchangeable and mean any disease, disorder, syndrome, anomaly, pathology, or abnormal condition of the podocytes or of the structure or function of their constituent parts.

The invention provides methods for treating podocyte related diseases or disorders comprising administering a calcimimetic compound to a subject in need thereof. In one aspect, the methods of the invention result in the shift of the balance of pro- and antiapoptotic factors so the pro-apoptotic factors dominate, podocytes do not undergo programmed cell death, and the number of podocytes does not decline or is restored.

In one aspect, the podocyte diseases or disorders treated by methods of the present invention stem from the perturbations in one or more functions of podocytes. These functions of podocytes include: (i) a size barrier to protein; (ii) charge barrier to protein; (iii) maintenance of the capillary loop shape; (iv) counteracting the intraglomerular pressure; (v) synthesis and maintenance of the glomerular basement membrane (GMB); (iv) production and secretion of vascular endothelial growth factor (VEGF) required for the glomerular endothelial cell (GEN) integrity.

Such disorders or diseases include but are not limited to loss of podocytes (podocytopenia), podocyte mutation, an increase in foot process width, or a decrease in slit diaphragm length. In one aspect, the podocyte-related disease or disorder can be effacement or a diminution of podocyte density. In one aspect, the diminution of podocyte density could be due to a decrease in a podocyte number, for example, due to apoptosis, detachment, lack of proliferation, DNA damage or hypertrophy.

In one aspect, the podocyte-related disease or disorder can be due to a podocyte injury. In one aspect, the podocyte injury can be due to mechanical stress such as high blood pressure, hypertension, or ischemia, lack of oxygen supply, a toxic substance, an endocrinologic disorder, an infection, a contrast agent, a mechanical trauma, a cytotoxic agent (cis-platinum, adriamycin, puromycin), calcineurin inhibitors, an inflammation (e.g., due to an infection, a trauma, anoxia, obstruction, or ischemia), radiation, an infection (e.g., bacterial, fungal, or viral), a dysfunction of the immune system (e.g., an autoimmune disease, a systemic disease, or IgA nephropathy), a genetic disorder, a medication (e.g., anti-bacterial agent, anti-viral agent, anti-fungal agent, immunosuppressive agent, anti-inflammatory agent, analgestic or anticancer agent), an organ failure, an organ transplantation, or uropathy. In one aspect, ischemia can be sickle-cell anemia, thrombosis, transplantation, obstruction, shock or blood loss. In on aspect, the genetic disorders may include congenital nephritic syndrome of the Finnish type, the fetal membranous nephropathy or mutations in podocyte-specific proteins, such as α-actin-4, podocin and TRPC6.

In one aspect, the podocyte-related disease or disorder can be an abnormal expression or function of slit diaphragm proteins such as podocin, nephrin, CD2AP, cell membrane proteins such as TRPC6, and proteins involved in organization of the cytoskeleton such as synaptopodin, actin binding proteins, lamb-families and collagens. In another aspect, the podocyte-related disease or disorder can be related to a disturbance of the GBM, to a disturbance of the mesangial cell function, and to deposition of antigen-antibody complexes and anti-podocyte antibodies.

In one aspect, the podocyte-related disease or disorder can be proteinuria, such as microalbumiuria or macroalbumiuria. In another aspect, the podocyte-related disease or disorder can be tubular atrophy.

A. Types of Podocyte Damage

Podocytes can be injured in a variety of diseases, resulting in the glomerular filtration barrier damage. Independent of the cause of podocyte injury, the early events leading to podocyte damage and disorders are characterized by molecular alterations of the slit diaphragm without visible morphological changes or by a reorganization of the foot process structure with fusion of filtration slits and apical displacement of the slit diaphragm.

The fate of the podocyte then depends on factors such as the persistence of the initial injury and/or reparative mechanisms. If the initial injury is halted and the reparative mechanisms are present, there may be resolution. See Shankland, S. J. (2006) Kidney Int. 69, 2131-2147. The fate of podocytes, such as survival or apoptosis, depends on the balance of pro- and antiapoptotic factors. If pro-apoptotic factors dominate, podocytes undergo programmed cell death, the number of podocytes declines. However, if the early structural changes in podocytes are not reversed, severe and progressive damage develops. This involves podocyte vacuolization, pseudocyst formation, and detachment of podocytes from the GMB, resulting in podocyte depletion. These events if unchanged may lead to the formation of synechiae via attachment of parietal epithelial cells of Bowman's capsule to denuded GBM areas.

One of the stereotypical reaction of podocytes to damage is a process called effacement, or change in podocyte shape, also referred to as fusion, retraction, or simplification. Effacement is characterized by gradual simplification of the inter-digitating foot process pattern, resulting in the formation of a flat and elongated looking cell. Effacement is believed to be due to retraction, widening and shortening of the processes of each podocyte, but it is not fusion of neighboring cells. Further, is it not specific to any single disease, but synonymous with different types of podocyte injury. Effacement was shown to start as a decrease in the degree of interdigitation by shortening and widening of foot processes, which is accompanied by degradation of some foot processes, followed by loss of the inter-digitating foot process pattern between individual cells. Foot process length may be reduced by up to 70%, and the width may decrease up to 60% compared to normal resulting in a flattened and spread out cell. Effacement is an active process that is energy dependent and is initiated by changes in the podocyte's cytoskeleton.

Another consequence of injury or damage to podocyte is a decrease in podocyte number, or podocytopenia. The etiology of podocytopenia includes apoptosis, detachment, and the inability or lack of podocytes to proliferate (see Table 1). Total podocyte number is a balance between proliferation and loss. Podocyte number can be reduced by either a decrease in proliferation due to lack of DNA synthesis, DNA damage or hypertrophy, and/or an increase in podocyte loss due to detachment and apoptosis.

Table 1 summarizes some common types of the podocyte injury and specific podocyte defects.

TABLE 1

| Type of podocyte injury | Specific podocyte defect |
|---|---|
| Slit diaphragm proteins | Nephrin mutation |
| | Podocyte mutation |
| | CD2AP haploinsufficiency |
| | FAT-1-targeted deletion |
| | Neph-1 deleteion |
| Reduced podocyte number | Detachment |
| | Apoptosis |
| | Lack of adequate proliferation |
| | DNA damage |
| | Hypertrophy |
| Podocyte effacement | Changes in slit diaphragm proteins |
| | Abnormal podocyte-GMB interactions |
| | (α, β dystroglycans, α3β1 integrin) |
| | Actin cytoscleleton reorganization |
| | due to synaptopodin, α-actinin-4, |
| | CDK5 |
| | Loss of negative charge |
| | Injury to apical membrane proteins |
| | (podocalyxin, NHERF2, Ezrin) |
| Loss of podocyte anion charge | Reduction of podocalyxin |
| | Reduction of glomerular epithelial |
| | protein (GLEPP) |
| Abnormal GBM | Proteases from podocyte |
| | Oxidants from podocyte |
| | GBM thickening due to matrix accumula- |
| | tion from podocyte |
| | Reduction of Heparan sulfate proteoglycan |
| Glomerular endothelial cell | Reduction of vascular endothelial |
| dysfunction | growth factor (VEGF) |

B. Causes of Podocyte Injury and Podocyte Response to Injury

Podocyte diseases or disorders can be classified according to their causes, e.g., congenital, hereditary and acquired causes. Acquired causes can be divided into immune and non-immune causes.

Congenital causes include abnormalities in structural podocyte proteins, such as in congenital nephritic syndrome of the Finnish type. This disorder is characterized by several mutations in nephrin leading to a loss of normal podocyte function, resulting in the onset of fetal proteinuria. Another congenital cause of cause of podocyte injury is the development of maternal antibodies to neutral endopeptidase and metallomembrane endopeptidase in mothers who are deficient in the enzyme. This gives rise to fetal membranous nephropathy.

Hereditary causes of podocyte injury typically include mutations in podocyte-specific proteins, such as α-actin-4, podocin and TRPC6. These mutations lead to hereditary proteinuria.

Acquired podocyte diseases can be immune and non-immune mediated. Examples of immune-mediated forms of podocyte injury include membranous nephropathy, minimal change disease and membranoproliferative glomerulonephritis associated with cryoglobulins. Non-immune causes of acquired podocyte injury include infectious causes such as HIV-associated nephropathy due to the local infection of podocytes by the HIV virus. It has been speculated that Pavro B19 virus may induce collapsing glomerulopathy in HIV-negative patients. Other examples of metabolic causes include diabetes, the metabolic syndrome and systemic hypertension, any cause of a reduced nephron number such as reflux nephropathy or chronic glomerulopathies, as well as infiltrative diseases of podocytes such as amyloid, where individual amyloid spicules “project” through the GBM, penetrating into the overlying podocytes.

Thus, podocytes can be injured by immune- and non-immune mediated diseases, resulting in damage to the glomerular filtration barrier. This typically results in proteinuria and effacement. Then the fate of the podocyte depends on several factors. If reparative mechanisms are present and the initial injury is halted, there may be resolution. However, if injury persists, and/or there are inadequate repair mechanisms, then proteinuria persists, leading to reduced renal function. The level of proteinuria can range from mild (<3 g/day) to nephritic (>3 g/day). Shankland (2006), supra.

### IV. Methods of Detection of Podocyte Abnormalities and Podocyte-Related Disorders and Diseases

In one aspect, early podocyte abnormalities can be detected using, for example, microscopy as described below. In the absence of a kidney biopsy, early diagnosis of podocyte-related diseases or disorders can be done on the basis of elevated excretion of protein (or albumin) into the urine. However, there are a lot of factors that affect the excretion of albumin in the urine, and a significant loss of podocyte to the point of inability to form a normal barrier is required.

One of the methods of detection of early podocyte damage is electron microscopy. Electron microscopy provides information about the presence and subcellular location of immune complexes (which are seen as electron-dense deposits), the degree of injury to glomerular cells, and the consistency of the basement membrane. Electron microscopy also detects fibrils and provides information on the ultrastructure of the kidney, such as podocyte effacement and flattening, which cannot be readily detected by light microscopy. Typical podocyte abnormalities include vacuolization, microcysctic or pseudocystic changes, the presence of cytoplasmic inclusion bodies, and detachment from the GBM. Others useful methods include light microscopy (e.g., to evaluate the shape of podocytes) and fluorescence microscopy (to localize and quantify stained proteins, e.g. proteins of the actin cytoskeleton).

Light microscopy describes glomerular cellularity, i.e., whether the number of glomerular cells is normal or increased (hypercellularity). Often light microscopy can distinguish which cell type (resident glomerular cells or infiltrating cells such as neutrophils) is increased; whether the GBMs are thickened and whether the capillary loops are patent, collapsed, or filled with material such as hyaline; and the presence or absence of glomerulosclerosis. Although the glomerulus is the primary site of injury in glomerular disease, the tubules and the interstitium must be carefully inspected because the degree of tubulointerstitial fibrosis is the best predictor of the prognosis in renal disease. The presence of glomerular crescents can also be detected on light microscopy. Crescents are layers of cells (parietal epithelial cells, podocytes, lymphocytes, and macrophages) in the Bowman space, and their presence signifies severe disease.

Immunofluorescent immunostaining determines the presence or absence of any underlying immune processes. Staining is directed against specific antibodies (e.g., IgG, IgA, and anti-GBM) and individual complement components (e.g., C3, C4, and C5b-9). The pattern of the immune components is also diagnostic. A granular pattern is typical of antigen-antibody complexes, such as in membranous nephropathy, whereas a linear pattern occurs in anti-GBM disease. The location of antibody or complement (e.g., in the mesangium in IgA nephropathy) also provides clues to the diagnosis. Immunostaining can determine the presence of matrix proteins (silver stain), amyloid fibrils (Congo red), and viral inclusions.

Disturbances in cultured podocyte functions can be studied by the use of activation, adherence, migration and proliferation assays. One indication of an early podocyte damage can be a disruption in the PINCH-1-ILK-α-parvin complex, resulting in the reduced podocyte-matrix adhesion, foot process formation or increase in apoptosis of podocytes. Another indicator of an early damage could be a disruption of function of synaptopodin, a member of a class of proline-rich actin associated proteins that are expressed in podocyte foot processes. It has been indicated that synaptopodin is essential for the integrity of the podocyte actin cytoskeleton and for the regulation of podocyte cell migration. See Yang et al, J Am Soc Nephrol. (2005) 16(7): 1966-76; Asanuma, K. et al, Nat Cell Biol. (2006) 8(5): 485-91; Pavenstadt et al, Physiol Rev. (2003) 83(1): 253-307. On the mRNA and protein level, specific podocyte genes including markers of cellular stress, apoptosis and specific proteins involved in the podocyte damage, can be studied as described in Tandon et al., Am J Physiol Renal Physiol. 2006, 17.; Durvasula R. V., Am J Physiol Renal Physiol. (2005) 289(3): F577-84. In blood and urine samples podocyte damage can also be assessed (see Hara et al., J Am Soc Nephrol. (2005) 16(2): 408-16; Vogelmann et al. (2003) Am J Physiol Renal Physiol. 285(1): F40-8, Pavenstadt et al, Physiol Rev. (2003) 83(1): 253-307).

Podocyte loss can be detected with a high degree of sensitivity by the abnormal presence in urine sediment of a gene selectively expressed in the podocyte so as to be podocyte-specific in the urinary tract. The methods of detection useful in the instant invention, are described in more detail in the publication WO 03/082202. Examples of markers useful for detection of podocyte damage include nephrin, glepp1, and Indian hedgehog. In one aspect, detection of a particular gene can be done using a reverse transcriptase quantitative polymerase chain reaction (RT-PCR), microarrays, Western blots, proteomics and in-situ hybridization, immunhisto- and immunocytochemistry. Other markers are podocin, FAT-1, CD2AP, Nephl, integrins, integrin-linked kinase, secreted protein acid rich in cysteine, Rho GTPases, α-actinin-4, synaptopodin, cyclin-dependent kinase5, podocalyxin, hic-5, TRPC6, dendrin, desmin, snail, notch, synaptopodin, HSP27, lamb4, podocalyxin, NHERF2, Ezrin, α, β dystroglycans, α3 β1 integrin collagen type 4, Wnt-4 and Hic-5, which can be detected from biopsy specimen, urine or blood analysis.

The following examples are offered to more fully illustrate the invention, but are not to be construed as limiting the scope thereof.

### EXAMPLE 1

This Example outlines methods used in the present invention.

Cell Culture

A well established cell model of conditionally immortalized murine podocytes was used to determine the impact of calcimimetics on podocyte function. These immortalized podocyte cell lines were generated from the Inmortomouse® (Charles River, Wilmington, Mass., USA) carrying a temperature-sensitive T-antigen as transgene, under the control of an interferon-γ inducible promoter. They were maintained cultured as previously described (Weins A et al. (2001) J Cell Biol 155: 393-404; Asanuma K. et al. (2005). J Clin Invest 115: 1188-1198. In brief, the cell line was maintained in RPMI 1640 (PAA Laboratories, Pasching, Austria) supplemented with 10% fetal bovine serum (PAA Laboratories, Pasching, Austria), 100 U/ml penicillin/streptomycin (Biochrom AG, Berlin, Germany) in humidified incubators with air-5% CO₂. In order to induce differentiation, podocytes were grown on collagen type I (BD Bioscience, Bedford, Mass., USA) under permissive conditions at 33° C. with IFN-γ (10 U/ml) (Roche, Mannheim, Germany) or under non-permissive conditions at 37° C. without IFN-γ for at least 10 days.

Tissue Extraction, Western Blotting, and Immunoprecipitation

Protein extraction from cultured podocytes and SDS-PAGE were done as previously described (Weins et al., supra). Total and phosphorylated proteins were detected by Western blotting as described previously, using the following primary antibodies: Bad (dilution 1:1000), pBad (1:1000), Bax (1:1000), Bid (1:1000), Bcl-xl (1:500), CREB (1:4000), ERK1/2 (1:4000), pERK 1/2 (1:4000), pJNK (1:1000), p38MAPK (1:500), p90RSK (1:2000) (Cell Signaling Technology, Beverly, Mass., USA), Bcl-2 (1:500), Caveolin-1 (1:500), 3-Actin (1:10000) (Abcam, Cambridge, UK) CaSR 1:100 (Alpha Diagnostics, San Antonio, Tex., USA). Western blot analysis was carried out according to standard protocols and visualized using enhanced chemiluminescence (ECL) immunoblot detection kits (Amersham Biosciences, Little Chalfont Buckinghamshire, UK).

Isolation of Nuclei and Cytoplasmatic Proteins

For isolation of nuclear and cytoplasmic proteins, the following protocol was used: cell lysate was dissolved in SDS-Lysis buffer (0.5% (w/v) SDS, 0.05 M Tris HCL, pH 8.0, 1 mM DTT), followed by 5 min sonification and 10 min incubation at 100° C. The mixture was centrifuged for 2 min at 14,000 g. The protein content of the supernatants was measured by the Bradford method (Protein Assay Kit; BioRad, Munich, Germany), and Western blot test was performed as described in Homme M. et al. (2003) Endocrinology 144: 2496-2504.

Immunofluorescence Microscopy

Immunofluorescence staining was performed on 4-μm frozen sections from rat kidney and cultured podocytes growing on collagen type I-coated glass cover-slips as described in Reiser J. et al. (2000) Kidney Int 57: 2035-2042 and Reiser J. et al. (2000). It has been shown that the glomerular slit diaphragm is a modified adherens junction. J Am Soc Nephrol 11: 1-8, 2000. To localize CaSR, a specific CaSR1-Antibody (1:100) (Alpha Diagnostics, San Antonio, Tex.), and Synaptopodin G¹ (1:200) (Gift from Peter Mundel, Mount Sinai, N.Y., USA) were used. The following secondary antibody was used: Alexa Fluor 488 IgG1+IgG3 1:800 (Molecular Probes, Invitrogen, Eugene, Oreg., USA) and Cy3 1:500 (Jackson ImmunoResearch Laboratories, West Grove, Pa., USA).

Preparation of Caveolin-Enriched Fraction

Caveolae-rich membrane fraction from podocytes was obtained using the sucrose gradient ultracentrifugation (Li, S. et al. (1995) J Biol Chem 270: 15693-15701). In brief, podocytes were homogenized in 2 ml of lysis buffer (10 nM Tris-Hcl, pH 7.5, 150 nM NaCl, 5 mM EDTA, 1 mM phenylmethysulfonyl fluoride, 10 μg/ml leupeptin, 10 μg/ml trypsin inhibitor, and 1% Triton X-100) followed by sonification. The homogenate was brought to 40% sucrose by addition of an equal volume of 80% sucrose and loaded in an ultracentrifuge tube. A discontinuous sucrose gradient was layered on top of the sample by placing 4 ml of 30% and 4 ml of 5% sucrose, respectively. After centrifugation at a speed of 200,000×G for 24 hrs at 40° C., twelve 1 ml fractions were collected and analyzed by Western Blot.

Flow Cytometric Detection of Apoptosis

All cells were treated as described, then washed and trypsinized. All washes and eluted cells were collected, pooled, and centrifuged. After washing cells with Annexin-binding buffer, cells were incubated with FITC-labelled Annexin-V (Molecular Probes) and propidium iodide (Molecular Probes) for 30 min. Annexin-V binds phosphatidylserine, which is translocated to the outer cell membrane during the initial stages of apoptosis. Propidium iodide was also applied to cells in order to distinguish necrosis from apoptosis. Cells were analyzed by flow cytometry (FACScalibur, Becton Dickinson). Apoptosis-associated fluorescence was measured using a log scale. Cells with high propidium iodide content were necrotic and were excluded from the analysis.

Real-Time RT-PCR

RNA was isolated with RNeasy Mini Kit (Qiagen, Hilden, Germany), checked for integrity on an agarose gel, and quantified photometrically (Biophotometer, Eppendorf, Hamburg, Germany). One μg of total RNA was reverse transcribed with oligo (dT)/random hexamer primers (1:10). Real time RT-PCR was performed with the ABI Prism 7000 sequence detection system (Applied Biosystems, Darmstadt, Germany) with specific primers for 18 s. Real-Time RT-PCR for mouse podocytes was performed using specific primers (MWG-Biotech AG, Ebersberg, Germany).

RNA Isolation and Oligonucleotide Microarray Hybridization

Total RNA was extracted from podocytes with the RNeasy Mini Kit (Qiagen, Hilden, Germany) and quality assayed using an Agilent 2100 Bioanalyzer (Agilent, Palo Alto, Calif., USA). Hybridization probe preparation and HG-U133A microarray processing were performed according to the standard protocols available from Affymetrix (Santa Clara, Calif., USA).

Microarray Data Analysis and Statistical Procedures

After various treatments cells were washed and trypsinized. All washes and eluted cells were collected, pooled, and centrifuged. Podocytes RNA was extracted using RNeasy Mini Kit (QIAGEN Inc. Valencia, USA). RNA samples, without evidence of degradation, were used for microarray analysis. Microarrays were performed with three independent mRNA samples per gene. Raw data from Affymetrix CEL files were normalized using the method described by Huber et al. (2002) Bioinformatics 18 Suppl 1:S96-104.

After normalization, probes from one probe set are summarized using the median polish function resulting in one value per probe set which is scaled to be on a log 2 scale. Statistical analysis was performed with the software package Micro Array Solution, version 1.3, from SAS (SAS Institute, Cary N.C.), using standard settings, except the following specifications: log-linear mixed models were fitted for values of perfect-matches (see Chu, T. et al. (2002) Math Biosci 176: 35-51.

Statistics

All experiments were performed al least 3 times, and samples were usually run in duplicate to account for technical and biological variability within and between experiments. Student's t-tests were used to compare experimental groups as appropriate. Data are given as mean ±SD. P<0.05 was considered significant.

### EXAMPLE 2

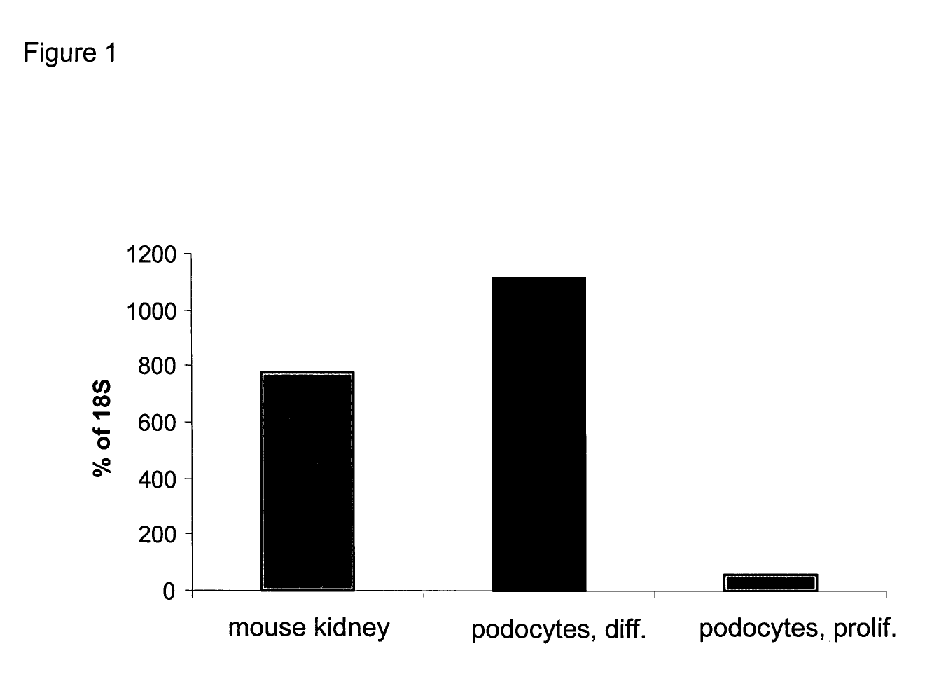

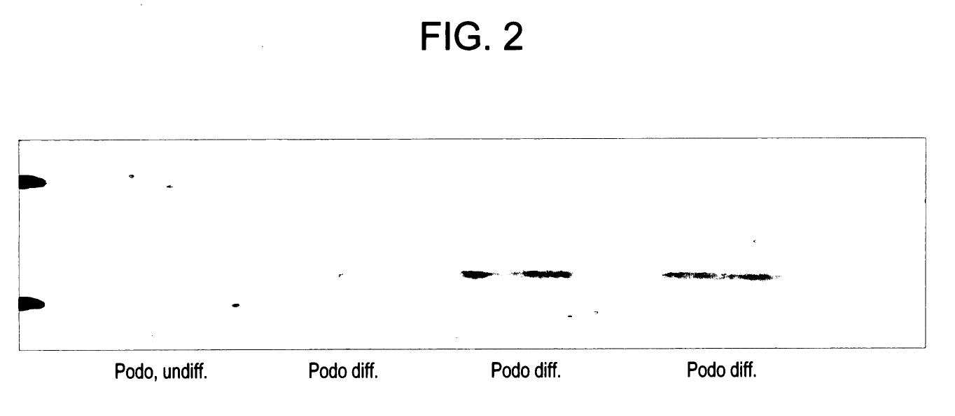

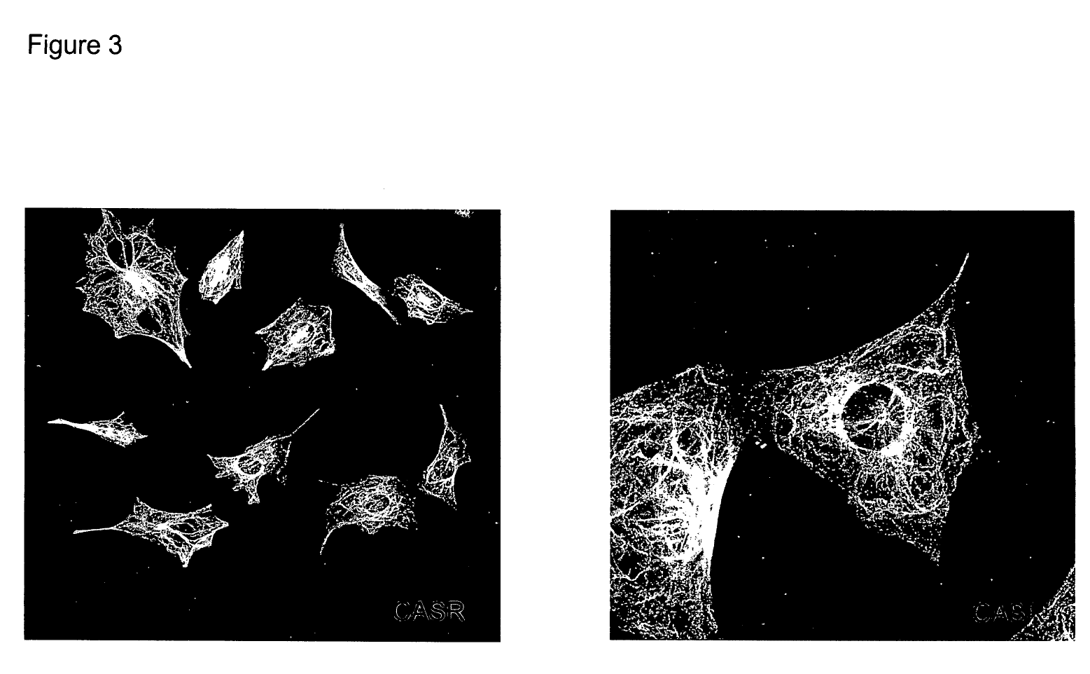

This Example demonstrates that the calcium sensor receptor is expressed in podocytes. FIGS. 1 and 2 illustrate the results of quantitative real-time PCR (rt-PCR) and Western immunoblotting, correspondingly. The conditionally immortalized murine podocytes expresses the CaSR-protein in the differentiated, but not in the undifferentiated, proliferating state. Additional immunohistochemical stainings were performed to identify CaSR in mouse podocytes (see FIGS. 1 and 2). FIG. 3 illustrates the results of Immunofluorescence staining and demonstrates that the CaSR is mainly expressed along cell-membranes, but also at cytoplasmatic filaments and around the nuclei and vesicular trafficking of the CaSR from the nucleus to the membrane.

### EXAMPLE 3

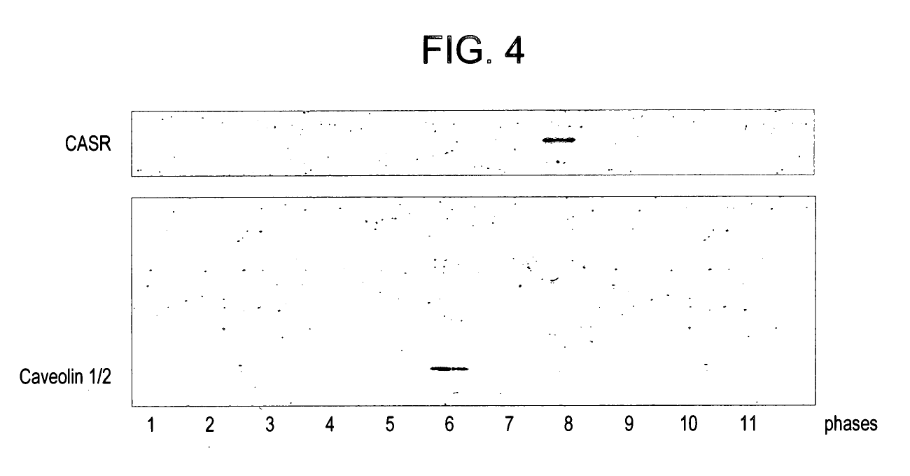

This Example demonstrates that the calcium sensor receptor is not found in the caveolin-1 enriched fraction. FIG. 4 illustrates the results of the Sucrose-gradient assay and demonstrates that CaSR proteins were restricted to caveolin-poor membrane fractions, suggesting that there is no direct local interaction of both proteins.

### EXAMPLE 4

This Example illustrates a specific cellular response of podocytes to calcimimetics. By using the calcimimetic Compound A (N-(3-[2-chlorophenyl]-propyl)-R-▪-methyl-3-methoxybenzylamine HCl) in concentrations of 4 nmol/l activation of intracellular proteins was demonstrated. Subsequent experiments were performed at concentrations of 10 to 50 nmol/l.

In mammalian cells, three extra-cellular signal activated mitogen-activated protein kinase (MAPK) cascades that execute complex cellular programs such as proliferation, differentiation and apoptosis, have been characterized. These cascades are (a) extra-cellular signal-regulated kinase (pERK), which is activated by growth factors, peptide hormones and neurotransmitters; (b) Jun kinase (JNK) and (c) p38 MAPK, which are both activated by cellular stress stimulus as well as growth factors.

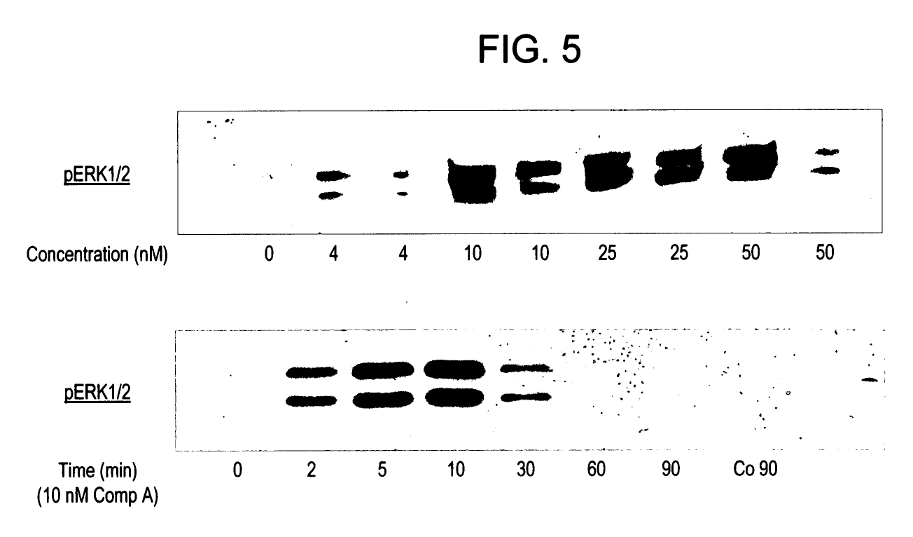

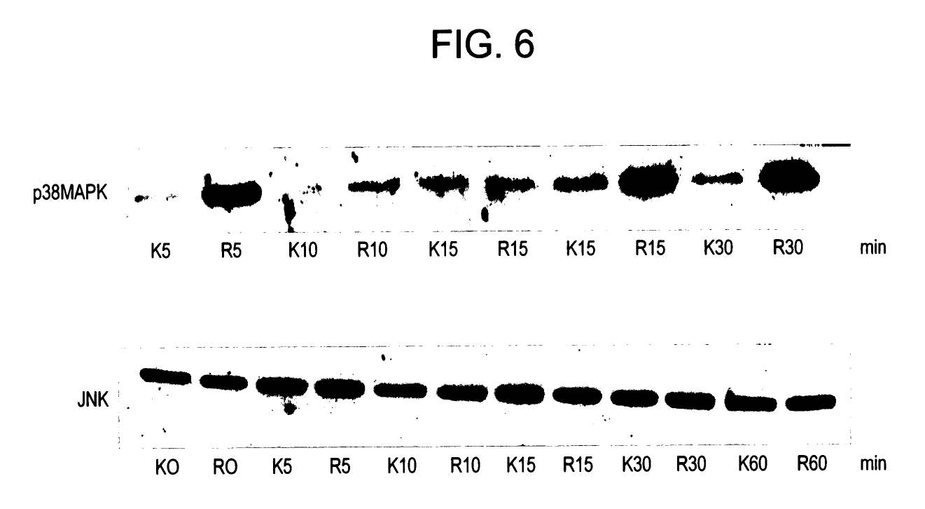

It has been established previously that the activation of the CaSR induces a variety of intracellular signalling pathways such as the protein kinase C(PKC) and the MAP kinase pathways. However, this Example for the first time demonstrates that in podocytes phosphorylation of ERK1/2 occurs in response to Compound A exposure in a dose—(4 to 50 nmol/l) and time-dependent manner (FIG. 5). With 10 mol/l of Compound A, a strong activation of ERK1/2 was observed, which could not further be increased by higher concentrations of Compound A. First phosphorylation steps were already detectable after 2 minutes of Compound A incubation, and maximal phosphorylation was found after 10 minutes (FIG. 5). p38 MAPK showed a clear biphasic response pattern to Compound A with an induced phosphorylation after 5 min and again after 30 and 60 min (FIG. 6, top panel). This biphasic phosphorylation was documented at a concentration of 10 nmol/l of Compound A. JNK showed no activation at any time-point in podocytes in response to Compound A (FIG. 6, bottom panel).

Next, the activation of downstream kinases and transcription factors such as 90 kDa ribosomal S6 kinases (p90RSK) was investigated. p90RSK is activated by MAPK in vitro and in vivo via phosphorylation. Phosphorylated p90RSK has been shown to translocate into the nucleus and to activate the transcription factor cAMP response element-binding protein (CREB) (Cataldi, A. et al. (2006) J Radiat Res (Tokyo) 47: 113-120; McCubrey, J. et al. (2000) Leukemia 14: 9-21). CREB is a bZIP transcription factor that activates target genes through cAMP response elements. The CREB multi-genic family is involved in cAMP signalling in cell proliferation, differentiation, apoptosis, survival, adaptive responses and in hematopoiesis (Cataldi et al., supra). CREB is able to mediate signals from numerous physiological stimuli and is activated by phosphorylation of ERK, Ca²⁺ and other stress signals.

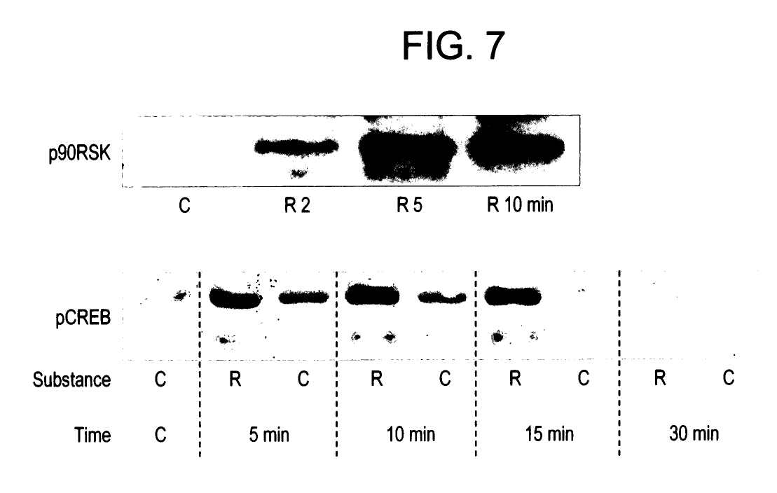

This Example further demonstrates that the downstream effector p90RSK is phosphorylated in a similar time pattern as ERK1/2 and leads to CREB phosphorylation (FIG. 7). This CREB activation is able to mediate signals from numerous physiological stimuli, resulting in the regulation of a broad array of cellular responses, such as regulating of apoptosis associated genes. Therefore downstream pro-survival factors were analyzed. The Bcl-2 family is involved in the regulation of apoptosis and exerts a survival function in response to a wide range of apoptotic stimuli through inhibition of mitochondrial cytochrome c release (Murphy, K. et al. (2000) Cell Death Differ 7: 102-111). It has been implicated in modulating mitochondrial calcium homeostasis and proton flux (Zhu, L., et al. (1999) J Biol Chem 274: 33267-33273). Unphosphorylated Bad is a pro-apoptotic member of the Bcl-2 family that can displace Bax from binding to Bcl-2 and Bcl-xL, resulting in cell death (Zha, J. et al. (1996) Cell 87: 619-628). Survival factors such as IL-3 can inhibit the apoptotic activity of Bad by activating intracellular signalling pathways that result in the phosphorylation of Bad (Zha, supra). Phosphorylation results in the binding of Bad to 14-3-3 proteins and the inhibition of Bad binding to Bcl-2 and Bcl-xL.

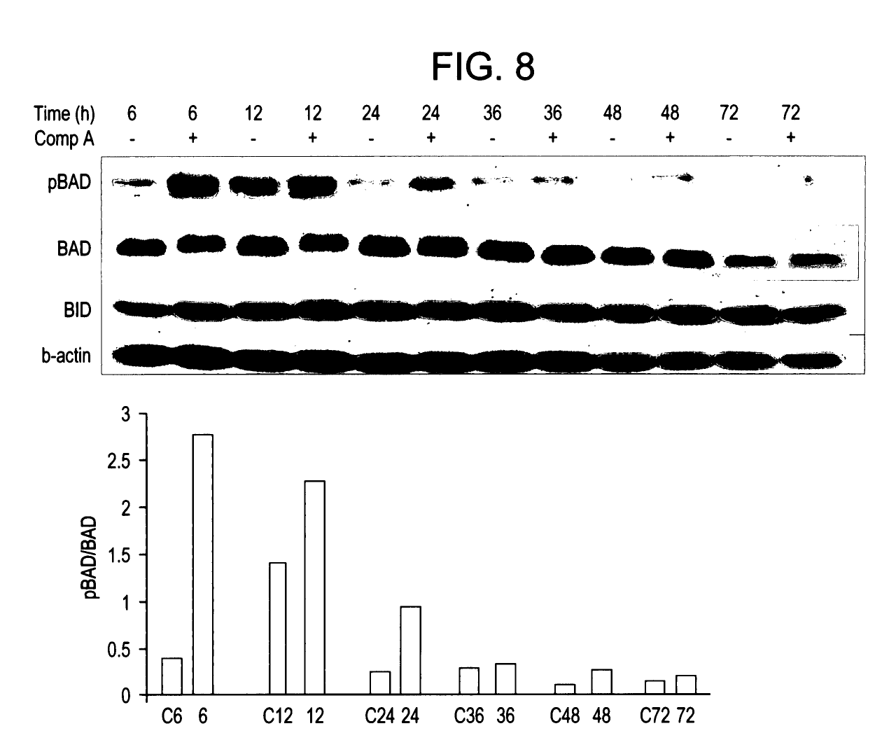

Results presented in FIG. 8 demonstrate that activation of the podocyte calcium sensing receptor by the calcimimetic Compound A clearly increases the amount of phosphorylated BAD in a time-dependent manner. Maximal phosphorylation occurs within 6 to 12 hours, the amount of non-phosphorylated BAD decreases at the same time (FIG. 8).

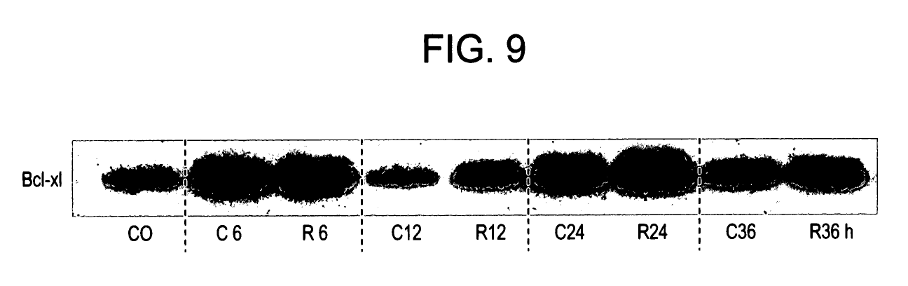

Bcl-xl, another Bcl-2 family member, is also induced in a time-dependent way by Compound A (FIG. 9). Other factors with pro-survival activity like BID are not affected by Compound A exposure, showing that only specific pro-survival pathways are turned on (FIG. 8), while the pro-apoptotic pathway mediated by BAD is turned off.

### EXAMPLE 5

This example illustrates inhibition of CREB-activation after the use of a specific MEK1/2-inhibitor.

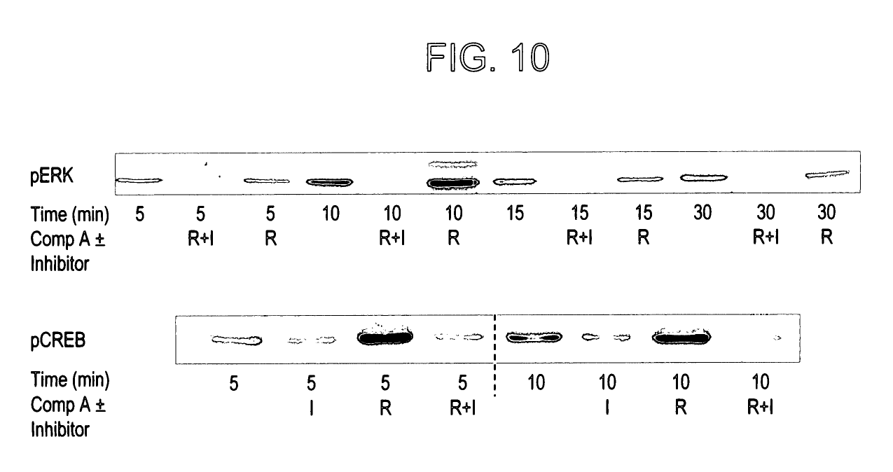

Results presented in FIG. 10 demonstrate that the administration of a specific MEK1/2-inhibitor (UO126, Cellsignaling, Boston, 10 μM), a kinase upstream of ERK1/2, was able to abolish completely the Compound A—induced phosphorylation of ERK1/2. In addition, U0126 suppressed the phosphorylation of CREB (FIG. 10). Blockade of MEK1/2 and MAP kinases can not be bypassed via activation of other pathways such as PKC. In summary, the calcimimetic Compound A activates intracellular signalling cascades which results in activation and up-regulation of proteins which have been shown to have clear pro-survival activity and in phosphorylation and subsequent inhibition of proapoptotic proteins such as BAD.

### EXAMPLE 6

This example illustrates the results of the microarray analysis of Compound A-dependent regulation of different podocytes genes.

The microarray analysis was performed in podocytes treated with 10 nmol/l of Compound A for 12 hrs and the findings were compared to respective untreated cells. 10% out of 15000 genes investigated were significantly regulated by Compound A in the podocytes. This finding suggests possibly a central role of the extracellular calcium sensing receptor in control of podocyte function. Table 2 summarizes the number of regulated genes in the functional group per number of genes analyzed.

TABLE 2

| | CaSR-regulated genes/number of genes |
|---|---|
| Genes involved in: | analyzed |
| Focal Adhesions | 30/174 |
| MAPK signaling pathways | 30/193 |
| Cytokine receptor interactions | 29/187 |
| Regulation of actin cytoskeleton | 26/171 |
| Adherens junction | 11/62 |
| Toll-like-receptor signaling | 10/69 |
| TGF beta signaling pathway | 9/62 |
| Wnt signaling pathways | 15/116 |
| Apoptosis | 9/72 |
| Nfkb signaling | 8/79 |
| Adhesion-extracellular matrix | 14/84 |

Of note, mRNA expression of pro-survival factor Bcl-2 was up-regulated, whereas pro-apoptotic genes calpain 1, calpain small subunit 1, caspase 7 and caspase 8 were down-regulated by Compound A. BAD, BID and Bcl-xl were not included in the array. In summary, the findings on mRNA level obtained by microarray technology indicate a central role of the CaSR in podocytes. They are in line with a pro-survival action of Compound A as already suggested by the analysis of the intracellular signaling cascades on protein level.

### EXAMPLE 7

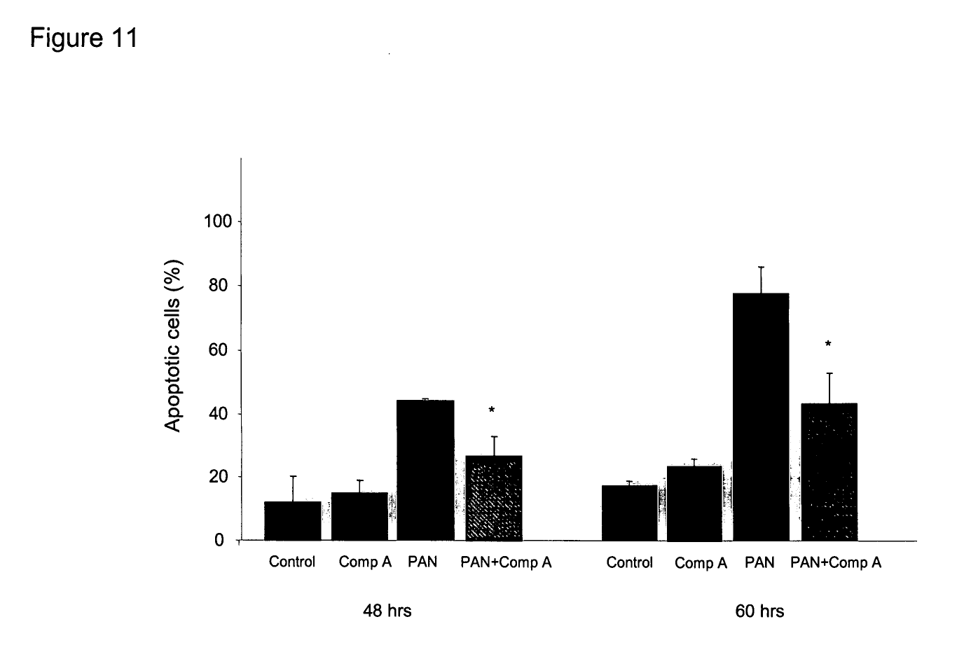

This example summarizes the results of FACS analyses. To prove that the Compound A—dependent induction of pro-survival genes is actually protecting podocytes from stress induced apoptosis, FACS analyses with podocytes exposed to puromycin aminonucleoside (PAN) were performed. PAN (30 μg/ml) increased apoptosis rate of cultured podocytes to 44 and 78% correspondingly after 48 and 60 hrs of incubation, as compared to a baseline apoptosis rate of less than 20% in respective controls (FIG. 11). Addition of Compound A (10 mmol/l) reduced PAN induced increase in apoptosis rate in three independent experiments by 55 and 60% after 48 and 60 hrs respectively (both p<0.05).

### EXAMPLE 8

This example illustrates that the calcimimetic Compound A provides significant protection from proteinuria in PAN-treated rats.

Puromycin aminonucleoside (PAN), which induces oxidant injury in cells via the xanthine oxidase pathway, has been used extensively as a model of podocyte injury. Podocyte injury in this model can be ameliorated by inhibitors of oxidants. Following PAN injection (15 mg/100 g body weight), injury in the rat podocytes is manifest by loss of interdigitating foot processes, detachment from the GBM, pseudocyst formation, reduction in anionic charge, attenuation of the underlying GBM, and associated leakiness of the glomerular filter resulting in proteinuria. The PAN model also progresses to a patchy glomerular scarring process [focal glomerulosclerosis (FSGS)]. FSGS in humans is commonly associated with progression to end-stage renal disease (ESRD) requiring renal replacement therapy. It has been observed that a single dose of PAN caused a limited reduction in podocyte number associated with minor glomerulosclerosis. Podocytes were stained against WT-1, a specific marker for podocytes and counted under an immunofluorescence microscope.

Animal Model of PAN Nephrosis

Male Sprague-Dawley rats weighing approximately 100 g were administered intraperitoneal PAN (Sigma Company, St. Louis, Mo., USA) at a dose of 15 mg/100 g body weight, or additionally at the same time a single injection of Compound A (doses 2 mg/100 g body weight) 4 hours before, or an equivalent volume of 0.9% saline (0.2-0.4 ml/animal) (N=10-15 animals per group). Urine was collected over a period of 24 hrs in metabolic cages at day −3, day 6 and at day 9. At day 9, the rats were euthanized, and kidneys were perfused with phosphate-buffered saline for two minutes, followed by paraformaldehyde in phosphate buffer for eight minutes at a pressure of 120 mm Hg. After perfusion, kidneys were quickly removed, and 3 to 4 mm sections of kidney were cut for fixation in formalin. Protein/Creatinin ratio was measured with ADVIA 2400, Bayer Diagnostics.

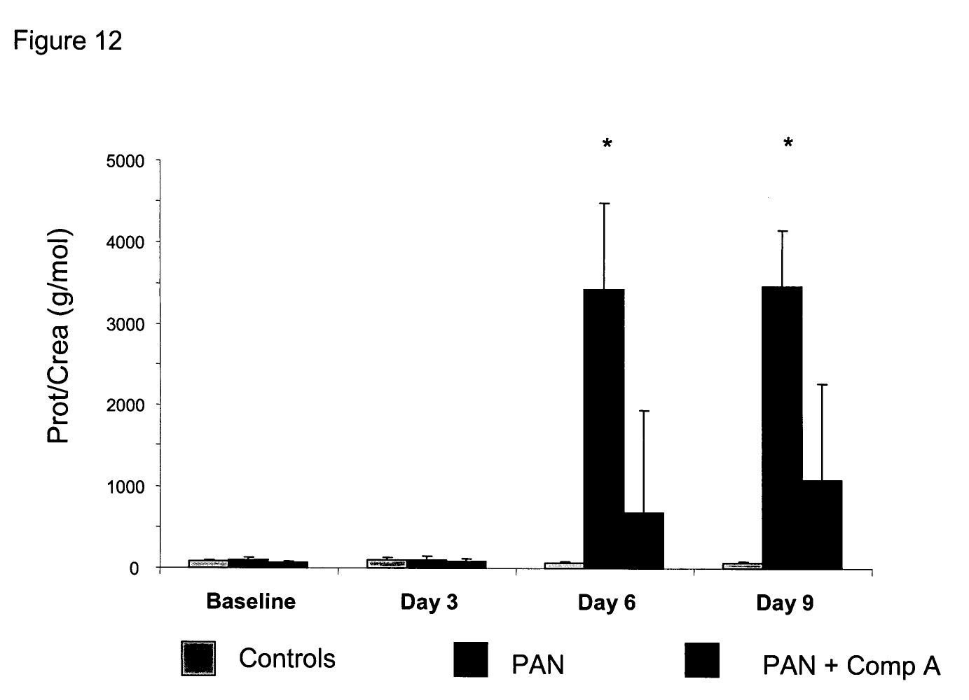

A single intraperitoneal injection of PAN (15 mg/100 g body weight) was used to assess the effect of Compound A on glomerular podocyte function and number (FIG. 12). Acute podocyte injury and proteinuria was induced as described in numerous prior studies. After a single doses of Compound A proteinuria was significantly induced in rats after day 6, compared to untreated controls rats, receiving saline. In the PAN and Compound A simultaneously receiving group proteinuria was successfully impeded. Thus, the results presented in FIG. 12 demonstrate that the calcimimetic Compound A is able to prevent the development of proteinuria in rats via the binding of Compound A to the podocytes.

All publications, patents and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method of treating a podocyte-related disease or disorder comprising administering a therapeutically effective amount of a calcimimetic compound to a subject in need thereof.

2. The method of claim 1, wherein the podocyte-related disease or disorder is podocytopenia.

3. The method of claim 1, wherein the podocyte-related disease or disorder is an increase in the foot process width or effacement.

4. The method of claim 1, wherein the podocyte-related disease or disorder a decrease in slit diaphragm length.

5. The method of claim 1, wherein the podocyte-related disease or disorder is a diminution of podocyte density.

6. The method of claim 1, wherein the podocyte-related disease or disorder is due to a podocyte injury.

7. The method of claim 6, wherein the podocyte injury is due to mechanical stress, ischemia, lack of oxygen supply, a toxic substance, an endocrinologic disorder, an infection, a contrast agent, a mechanical trauma, a cytotoxic agent, a medication, an inflammation, radiation, an infection, a dysfunction of the immune system, a genetic disorder, an organ failure, an organ transplantation, or uropathy.

8. The method of claim 7, wherein the infection is bacterial, fungal, or viral.

9. The method of claim 7, wherein the inflammation is due to an infection, a trauma, anoxia, obstruction, or ischemia.

10. The method of claim 7, wherein the dysfunction of the immune system is an autoimmune disease, a systemic disease, or IgA nephropathy.

11. The method of claim 7, wherein the cytotoxic agent is cis-platinum, adriamycin, puromycin or a calcineurin inhibitor.

12. The method of claim 7, wherein the medication is an anti-bacterial, anti-viral, anti-fungal, immunosuppressive, anti-inflammatory, analgetic or anticancer agent.

13. The method of claim 7, wherein the ischemia is sickle-cell anemia, thrombosis, transplantation, obstruction, shock or blood loss.

14. The method of claim 7, wherein the genetic disorder is congenital nephritic syndrome of the Finnish type, the fetal membranous nephropathy or a mutation in podocyte-specific proteins.

15. The method of claim 1, wherein the podocyte-related disease or disorder is due to an abnormal expression or function of nephrin, podocin, FAT-1, CD2AP, Neph1, integrins, integrin-linked kinase, secreted protein acid rich in cysteine, Rho GTPases, α-actinin-4, synaptopodin, cyclin-dependent kinase5, podocalyxin, hic-5, GLEPP, TRPC6, dendrin, desmin, snail, notch, synaptopodin, HSP27, lamb4, podocalyxin, NHERF2, Ezrin, α, β dystroglycans, α3 β1 integrin collagen type 4 or Wnt-4.

16. The method of claim 1, wherein the disease is proteinuria.

17. The method of claim 16, wherein the proteinuria is microalbumiuria.

18. The method of claim 16, wherein the proteinuria is macroalbumiuria.

19. The method of claim 1, wherein the disease is tubular atrophy.

20. The method of claim 1, wherein the calcimimetic compound is a compound of the Formula I 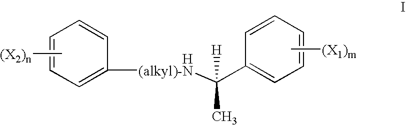
wherein:
X₁ and X₂, which may be identical or different, are each a radical chosen from CH₃, CH₃O, CH₃CH₂O, Br, Cl, F, CF₃, CHF₂, CH₂F, CF₃O, CH₃S, OH, CH₂OH, CONH₂, CN, NO₂, CH₃CH₂, propyl, isopropyl, butyl, isobutyl, t-butyl, acetoxy, and acetyl radicals, or two of X₁ may together form an entity chosen from fused cycloaliphatic rings, fused aromatic rings, and a methylene dioxy radical, or two of X₂ may together form an entity chosen from fused cycloaliphatic rings, fused aromatic rings, and a methylene dioxy radical; provided that X₂ is not a 3-t-butyl radical;
n ranges from 0 to 5;
m ranges from 1 to 5; and
the alkyl radical is chosen from C₁-C₃ alkyl radicals, which are optionally substituted with at least one group chosen from saturated and unsaturated, linear, branched, and cyclic C₁-C₉ alkyl groups, dihydroindolyl and thiodihydroindolyl groups, and 2-, 3-, and 4-piperidinyl groups;
or a pharmaceutically acceptable salt thereof.

21. The method of claim 20, wherein the calcimimetic compound is N-(3-[2-chlorophenyl]-propyl)-R-α-methyl-3-methoxybenzylamine or a pharmaceutically acceptable salt thereof.

22. The method of claim 1, wherein the calcimimetic compound is a compound of the Formula II 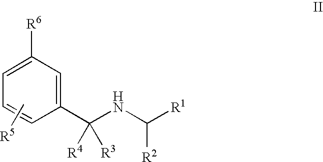
wherein:
R¹ is aryl, substituted aryl, heterocyclyl, substituted heterocyclyl, cycloalkyl, or substituted cycloalkyl;
R² is alkyl or haloalkyl;
R³ is H, alkyl, or haloalkyl;
R⁴ is H, alkyl, or haloalkyl;
each R⁵ present is independently selected from the group consisting of alkyl, substituted alkyl, alkoxy, substituted alkoxy, halogen, —C(═O)OH, —CN, —NR^{d}S(═O)ₘR^{d}, —NR^{d}C(═O)NR^{d}R^{d}, —NR dS(═O)ₘNR^{d}R^{d}, or —NR^{d}C(═O)R^{d};
R⁶ is aryl, substituted aryl, heterocyclyl, substituted heterocyclyl, cycloalkyl, or substituted cycloalkyl;
each R^{a} is, independently, H, alkyl or haloalkyl;
each R^{b} is, independently, aryl, aralkyl, heterocyclyl, or heterocyclylalkyl, each of which may be unsubstituted or substituted by up to 3 substituents selected from the group consisting of alkyl, halogen, haloalkyl, alkoxy, cyano, and nitro;
each R^{c} is, independently, alkyl, haloalkyl, phenyl or benzyl, each of which may be substituted or unsubstituted;
each R^{d} is, independently, H, alkyl, aryl, aralkyl, heterocyclyl, or heterocyclylalkyl wherein the alkyl, aryl, aralkyl, heterocyclyl, and heterocyclylalkyl are substituted by 0, 1, 2, 3 or 4 substituents selected from alkyl, halogen, haloalkyl, alkoxy, cyano, nitro, R^{b}, —C(═O)R^{c}, —OR^{b}, —NR^{a}R^{a}, —NR^{a}R^{b}, —C(═O)ORC₁—C(═O)NR^{a}R^{a}, —C(═O)R^{c}, —NR^{a}C(═O)R^{c}, —NR^{a}S(═O)ₙR^{c} and —S(═O)ₙNR^{a}R^{a};
m is 1 or 2;
n is 0, 1 or 2; and
p is 0, 1, 2, 3, or 4;
provided that if R² is methyl, p is 0, and R⁶ is unsubstituted phenyl, then R¹ is not 2,4-dihalophenyl, 2,4-dimethylphenyl, 2,4-diethylphenyl, 2,4,6-trihalophenyl, or 2,3,4-trihalophenyl;
or a pharmaceutically acceptable salt thereof.

23. The method of claim 1, wherein the calcimimetic compound is cinacalcet HCl.

24. The method of claim 1, wherein the calcimimetic compound is a compound of the Formula III 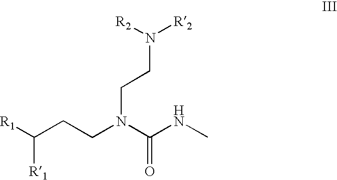
R₁ and R′₁, which may be the same or different, represent an aryl radical, a heteroaryl radical, an aryl or heteroaryl radical substituted by one or more halogen atoms, by one or more hydroxy groups, by one or more linear or branched alkyl or alkoxy radicals containing from 1 to 5 carbon atoms, by one or more trifluoromethyl, trifluoromethoxy, —CN, —NO₂, acetyl, carboxyl, carboalkoxy or thioalkyl groups and the oxidised sulfoxide or sulfone forms thereof, thiofluoroalkoxy groups,
or R₁ and R′₁, form, with the carbon atom to which they are linked, a cycle of formula: 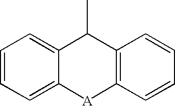
in which A represents a single bond, a —CH₂— group, an oxygen, nitrogen or sulfur atom,
R₂ and R₁₂ form, with the nitrogen atom to which they are linked, a saturated heterocycle containing 4 or 5 carbon atoms optionally substituted by one or more linear or branched alkyl radicals containing from 1 to 5 carbon atoms, said heterocycle optionally containing a further heteroatom, itself being optionally substituted by a radical R₅ in which R₅ represents a hydrogen atom, a linear or branched alkyl radical containing from 1 to 5 carbon atoms, optionally substituted by an alkoxy or acyloxy radical,
or R₂ and R₁₂, which may be the same or different, represent a hydrogen atom, a linear or branched alkyl radical containing from 1 to 5 carbon atoms optionally substituted by a hydroxy or alkoxy radical containing from 1 to 5 carbon atoms,
R₃ represents a thiazolyl, oxazolyl, benzothiazolyl or benzoxazolyl group of formula: 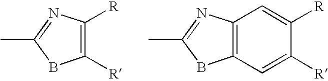
in which B represents an oxygen atom or a sulfur atom, in which R and R′, which may be the same or different, represent a hydrogen atom, a halogen atom, a hydroxy radical, a trifluoromethyl radical, a trifluoromethoxy radical, alkyl, alkoxy, alkoxycarbonyl or alkylthio radicals and the oxidised sulfoxide and sulfone form thereof linear or branched containing from 1 to 5 carbon atoms, an aryl or heteroaryl radical, an aryl or heteroaryl radical substituted by one or more groups selected from a halogen atom, a linear or branched alkyl radical containing from 1 to 5 carbon atoms, a trifluoromethyl radical, a trifluoromethoxy radical, a —CN group, an amino, dialkylamino and —NH—CO-alkyl group, an alkylthio group and the oxidised sulfoxide and sulfone form thereof, an alkylsulfonamide —NH—SO₂-alkyl group or by a morpholino group,
or R and R′ on the thiazolyl or oxazolyl group can form a saturated or unsaturated cycle comprising or not comprising one or more optionally substituted heteroatoms,
or a pharmaceutically acceptable salt thereof.

25. A method of treating a podocyte-related disease or disorder comprising administering a therapeutically effective amount of a pharmaceutical composition comprising a calcimimetic compound together with a pharmaceutically acceptable carrier to a subject in need thereof.
